(19) United States
(12) Plant Patent Application Publication
(10) Pub. No.: US 2002/0059664 P1
(43) Pub. Date: May 16, 2002

(54) GRANDIFLORA ROSE PLANT NAMED 'MACSUPBOW'

(76) Inventor: SAM MCGREDY, AUCKLAND (NZ)

Correspondence Address: CHRISTIE PARKER & HALE, P O BOX 7068, PASADENA, CA, 911097068

(21) Appl. No.: 09/257,879

(22) Filed: Mar. 2, 1999

(51) Int. Cl. A01H 5/00

(52) U.S. Cl. PLT/137

(57) ABSTRACT

A new variety of Grandiflora rose suitable for garden decoration, having flowers of a pale, silvery lavender coloration.

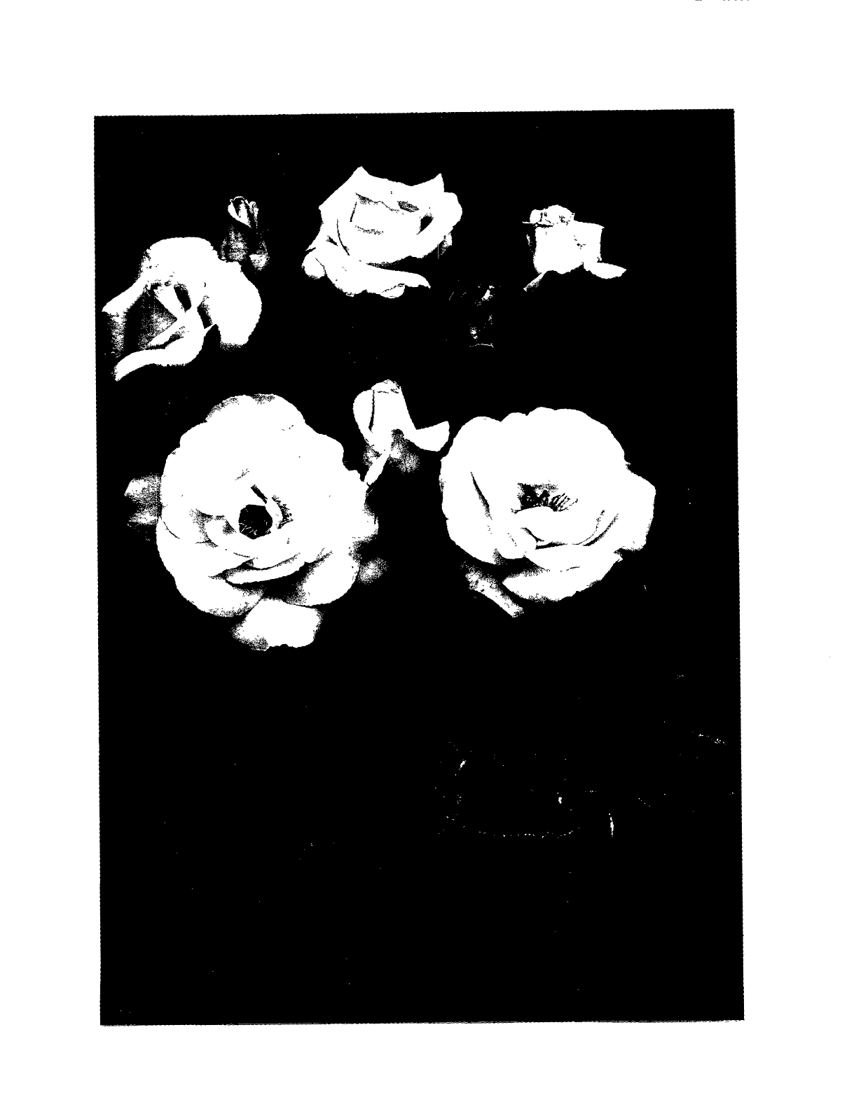

### BACKGROUND OF THE INVENTION

This invention relates to a new and distinct variety of Grandiflora Rose. The varietal denomination of the new variety is ‘Macsupbow’. It has as its seed parent the variety known as ‘Kortember’ and as its pollen parent the variety known as ‘Arolaqueli’.

### SUMMARY OF THE INVENTION

Among the features which distinguish the new variety from other presently available and known commercial rose cultivars are the following combination of characteristics: its attractive lavender pink color, its strong petal substance, its deep green, close set foliage. ‘Macsupbow’ may be asexually propagated by cuttings, budding, grafting, layering or tissue culture.

The new variety displays flowers of pure, pale, silvery lavender color. The high centered nicely spiraled blooms are borne in considerable profusion and coupled with an intense fragrance, make it a good garden subject as well as a source of fragrant, nearly thornless cut blooms for the house.

The plant itself is bushy and free branching. Although not as tall as some grandifloras, its bloom size coupled with large basal candelabras in late summer and fall place it well within the confines of the grandiflora class.

Asexual reproduction of the new variety by budding as performed in Kern County, Calif., and Clackamas County, Oreg. shows that the foregoing and other distinguishing characteristics come true to form and are established and transmitted through succeeding propagations.

### COMPARISON WITH PARENTS

The new rose may be distinguished from its seed parent, ‘Kortember’ by the following combination of characteristics: ‘Macsupbow’ is a light lavender pink grandiflora with large flowers while ‘Kortember’ is a deep salmon (orange-pink) flowered hybrid tea.

The new variety may be distinguished from its pollen parent, ‘Lagerfeld’, (‘Arolaqueli’) by the following combination of characteristics: ‘Lagerfeld’, is deeper in color, has more petals, (30); and tends to be a taller plant clothed in non-lustrous matte green foliage.

### BRIEF DESCRIPTION OF ILLUSTRATION

The accompanying photograph illustrates specimens of the new variety and shows the flowering thereof from bud to full bloom depicted in color as nearly correct as it is possible to make in a color illustration of the character. Throughout this specification, color values are based upon the Colour Chart of The Royal Horticultural Society of London, England, except where common terms of color definition are employed.

### DESCRIPTION OF THE NEW VARIETY

The following description is of rose plants of the new cultivar grown outdoors in Wilsonville, Oreg. in the month of September. Phenotypic expression may vary with environmental, cultural and climatic conditions, as well as differences in conditions of light and soil.

### FLOWER

The new variety bears its flowers sometimes singly, usually in clusters of three to five or more per stem. Flowers are borne in irregular flat clusters on strong long stems (about 40 to about 60 cms). Outdoors, the plant blooms freely and continuously during the growing season. The flowers have a strong penetrating tea to apple, spicy fragrance.

### BUD

The peduncle is of average to long length for the class (about 10 to about 12 cms.), of average to heavy caliper, and usually erect. It is almost entirely smooth, with very few stipitate glands, and very few hair and prickles. Peduncle color is between 146B and 146C.

Before the calyx breaks, the bud is of medium size for the class (about 2.4 to about 2.6 cms. in diameter at the widest point), moderately short to medium in length (about 2.8 to about 3.2 cms.), and pointed in shape with a conspicuous neck. The surface of the bud bears few foliaceous appendages and stipitate glands and glandular bloom usually without foliaceous parts extending beyond the tip of the bud. Bud color is between 146C and 148C.

The inner surface of the sepals is covered and edged with very fine wooly tomentum; sepal margins are lined with some stipitate glands and hairs.

As the petals open (after the calyx breaks), the bud is medium for the class about 4 to about 4.5 cms. in diameter at the widest point, about 5.5 to about 6.5 cms. in length, and pointed in form. The color of the under surfaces of the newly opened petals is between 65B and 65C. The color of the upper surfaces of the newly opened petals is between 69D and 65D. The bud opens up well and is generally not retarded from opening by wet, cold weather.

### BLOOM

When fully open, the bloom ranges from about 12 to about 14 cms. in diameter. Petallage is double with about 20 to 24 petals and 5 to 20 petaloids arranged regularly. When partially open, the bloom form is moderately high centered, and the petals are somewhat loosely spiraled with petal edges rolled outward. When fully open, the bloom form is somewhat flat to cupped, and the petals are loosely imbricated with petal edges rolled outward.

### PETALS

The substance of the petals is moderately crisp and thin to medium thickness, with upper surfaces moderately satiny and under surfaces slightly shiny. The outer petals are round to deltoid in shape with apices rounded to flat. The inner petals are broadly round in shape with apices somewhat rounded to flat.

### NEWLY OPENED FLOWER

The under and upper surface of the outer petals is near 69D.

The under and upper surface of the intermediate petals is near 65D.

The under and upper surface of the inner petals is near 65D.

The general tonality of the newly opened flower is near 65D.

### THREE DAY OLD FLOWER

The under and upper surface of the outer petals is near 69D.

The under and upper surface of the inner petals is near 69D.

The general tonality of the three day old flower is near 69D. Stamens generally are visible on fully opened blooms.

On the spent bloom, the petals usually drop off cleanly, fading to near 69D, and are affected by cold, wet, weather exhibiting rainspotting and reddish discoloration.

In September in Wilsonville, Oreg., blooms on the bush growing outdoors generally last two to three days. Cut roses from plants grown outdoors and kept at normal indoor living temperatures generally last two to three days.

### MALE REPRODUCTIVE ORGANS

Stamens are few to average and are arranged irregularly about the pistils. The filaments are somewhat long length, few without anthers. Filaments are near 16B in color. The anthers are somewhat small for the class and all open approximately at the same time. Anther color is between 16B and 16C when immature and between 16A and 16B at maturity. Pollen is somewhat sparse and near 16A in color.

### FEMALE REPRODUCTIVE ORGANS

Pistils vary in number (approximately 75-100). The styles are moderately even, average in length, moderately thin to average in caliper, and loosely bunched. Stigma color is between 11B and 11C. Style color is near 11D. Ovaries are usually enclosed in the calyx, although some may protrude from the calyx at the outer edge.

No hips have been observed on this variety when grown in Wilsonville, Oreg.

### FOLIAGE

The compound leaves are usually comprised of three to five leaflets and are borne abundantly. The leaves are about 18 to about 20 cms. in length and about 15 to about 18 cms. in width at the widest point, very leathery in texture, and semiglossy to matte in finish. The leaflets are about 8 cms. in length and about 5 to about 6 cms. in width at the widest point, shaped oblong moderately acute apices and somewhat round bases. Their margins are usually simply serrate.

The upper surface of the mature leaf is between 137A and 139A. The under surface of the mature leaf is between 136C and 137D. The upper and under surface of the young leaf is near 144A, sometimes heavily suffused with between 59A and 59B. The under surface of the young leaf is between 153B and 153C, sometimes heavily suffused with between 59D and 60D.

The rachis is average to heavy in caliper and moderately smooth. The upper side is deeply grooved with few hairs and prickles on the edges of the grooves. The under side of the rachis is very moderately smooth with few hairs and very small prickles.

The stipules are about 1.5 to about 1.7 cms. in length with somewhat narrow to medium width to long straight points that usually turn out at an angle of more than 45 degrees.

The plant displays an above average degree of resistance to powdery mildew and blackspot and rust as compared to other commercial varieties grown under comparable conditions in Wilsonville, Oreg.

### GROWTH

The plant has a bushy upright medium to tall growth habit (about 150 to about 200 cms. in height and about 75 to about 100 cms. spread at the widest point), with moderately full branching. It displays somewhat vigorous growth and the canes are of medium to heavy caliper for the class.

The color of the major stems is between 137D and 138A. They bear few large prickles that are about 0.4 to about 0.6 cms in length. The large prickles are hooked slightly downward with a somewhat short to medium length and a long narrow base; prickle color is between 165B and 165C. The major stem bears no small prickles. There are no hairs.

The color of the branches is between 137B and 138A. They bear few large prickles that are of similar size and shape to the large prickles on the major stems; prickle color is between 163C and 163D. The branches bear no small prickles. There are no hairs.

The color of the new shoots is between 144A and 144B often heavily suffused with between 59D and 60D. They bear few large prickles that are of similar size and shape to the large prickles on the major stems; prickle color is between 59C and 60C. The shoots bear no small prickles. There are no hairs.

I claim:

1. A new and distinct Grandiflora rose plant of the variety substantially as described and illustrated herein.
